(19) United States
(12) Patent Application Publication
(10) Pub. No.: US 2025/0205058 A1
(43) Pub. Date: Jun. 26, 2025

(54) ELBOW PROSTHESIS IMPLANT

(71) Applicants: SEMMELWEIS EGYETEM, BUDAPEST (HU); BUDAPESTI MUSZAKI ÉS GAZDASÁGTUDOMÁNYI EGYETEM, BUDAPEST (HU)

(72) Inventors: György KOCSIS, BUDAPEST (HU); Gábor SZEBÉNYI, VECSÉS (HU); Tamás TEMESI, BUDAPEST (HU)

(21) Appl. No.: 18/852,102

(22) PCT Filed: Apr. 12, 2023
(86) PCT No.: PCT/HU2023/050018

§ 371 Date: Sep. 27, 2024

(30) Foreign Application Priority Data
Apr. 13, 2022 (HU) P2200117

(51) Int. Cl. A61F 2/38; A61F 2/30

(52) U.S. Cl. CPC A61F 2/3804; A61F 2002/30375; A61F 2002/30451; A61F 2002/30492

(57) ABSTRACT

The invention relates to an elbow prosthesis implant having a forearm stem (1) an upper arm stem (2), and a shaft (3) fixed to the upper arm stem (2) hingedly connecting the stems (1, 2) to each other, and an insert (9) is arranged between the shaft (3) and an inner surface (1c) of a hole (1a). The insert has an axial hole (10), and an outer cover (4) is attached to the insert (9), on which first (31) and second covering plates (32) are oppositely arranged. and a forearm stem opening (43) is formed, and the flange (31p, 32p) of the holes (31f, 32f) is welded to the flange (10p) of the axial hole (10), the flange (43p) of the opening (43) of the forearm stem (1) is welded to the forearm stem (1) along the respective flanges (31p, 32p, 10p, 43p) by a continuous scam.

This invention relates to an elbow prosthesis implant having a forearm stem formed with a head having a hole, an upper arm stem, and a shaft fixed to the upper arm stem and passing through the hole of the forearm stem as well as hingedly connecting the legs to each other, and an insert of the form of a pipe section is arranged between the shaft and the inner surface of the hole, the insert having an axial hole receiving the shaft.

One of the main problems of orthopaedic endoprosthesis systems is the wear of contact surfaces. Abrasion partially destroys the carefully designed fit of the surfaces, and partially leads to the formation of wear products. The geometry of the movement of the prosthesis and the load vectors can change leading to early loosening. The creation of wear products is problematic because, through the activation of the immune system and the RANK ligand pathway, it causes opsonic effect by stimulating bone phagocytes to work intensively. The result of the latter is that the bone is resorbed near the implanted endoprosthesis, causing the prosthesis to loosen.

Accordingly, the wear surfaces of the prostheses are designed with great care. Medical steel—polyethylene, titanium-polyethylene, ceramic-polyethylene and ceramic—ceramic surface pairs are common nowadays.

The common feature of the currently most commonly used elbow prosthetic systems is that they use metal prosthetic stems in both the forearm and upper arm, as well as polyethylene inserts to create wear surfaces. Due to their tendency to creak and the fragility of the edges, as well as mounting difficulties, ceramic inlays are currently not widely used. Polyethylene wear pads are usually attached to metal components with a detachable and/or built-up bond. So-called backside wear attracts increasing attention in the last decade. In this process, the polyethylene insert wears not only on the surface intended for wear, but also in the area of the contact metal surface that secures the insert. The latter can reach about 30% of the total wear in the case of a knee prosthesis. Backside wear is also present in the case of elbow prostheses, it can be observed on explanted prosthetic components, but no quantitative measurement is known to determine this phenomenon.

In the case of the three most common elbow prosthesis systems, the polyethylene components are attached to the surface of the metal prosthesis elements by forming some form-locking bond, very similarly to the knee prosthesis and other prosthesis systems. In the case of the latter, backside wear is common, as the form-locking joints do not prevent the wear product formed on the metal-polyethylene interface from forming and leaving to the surrounding tissues.

U.S. Pat. No. 8,926,705 B2 publication document therefore describes a multi-component joint prosthesis that is built into end surfaces prepared and formed between a first bone and a second bone opposite to it. The first component is attached to the reconstructed end surface of the first bone and has a first free bearing surface. The second component is attached to a machined end surface of at least one of the radius and ulna and has a second exposed support surface. The intermediate component between the first and second anchored components is hingedly supported. Several chambers for capturing microshiver from the wear of the artificial joint are formed in the joint, which communicate with at least one of the joint surfaces to isolate and capture the shiver from the joint wear. The disadvantage of this solution is that the openings of the chambers break the continuous joint surface, and the shiver damages the joint surface in this case as well, until it reaches one of the chambers. At the same time, the chambers used on the back side are not suitable for capturing shiver, since the movement of the back side and thus the transmission of shiver in the direction of the chambers is limited, which leaves the prosthesis eventually and initiates the intensive intervention of phagocytes.

Our object by developing the elbow prosthesis according to the invention is to prevent shiver from wear of the back surface from escaping from the prosthesis.

We achieved our goal by developing an elbow prosthesis implant having a forearm stem formed with a head having a hole, an upper arm stem, and a shaft fixed to the upper arm stem and passing through the hole of the forearm stem as well as hingedly connecting the legs to each other, and an insert in the form of a pipe section is arranged between the shaft and the inner surface of the hole, the insert having an axial hole receiving the shaft, and an outer cover covering the head is attached to the insert, on which a first covering plate with a first hole and a second covering plate with a second hole both having the same diameter as the diameter of the axial hole of the insert) formed as a pipe section are oppositely arranged, and a forearm stem opening is formed, and the flange of the holes is welded to the flange) of the axial hole, the flange of the opening of the forearm stem is welded to the forearm stem along the respective flanges by a continuous seam.

The outer cover and one of the covering plates and advantageously the insert as well are formed from a single piece, and the other covering plate is welded to the cover and the insert.

The first covering plate and the second covering plate are disc-shaped elements.

The shaft is fixed in the shaft grooves formed on the upper arm stem.

The shaft is fixed with a locking pin in the shaft grooves formed on the upper arm stem.

The outer cover and the insert are made of UHMWPE or HxUHMWPE material, and the stems are made of a medical metal alloy.

A first skirt surface is formed on the outer cover, which is in contact with a skirt surface of the upper arm stem being congruent and parallel to the first skirt surface.

The invention will be described in detail below with reference to the attached drawing. In the drawing:

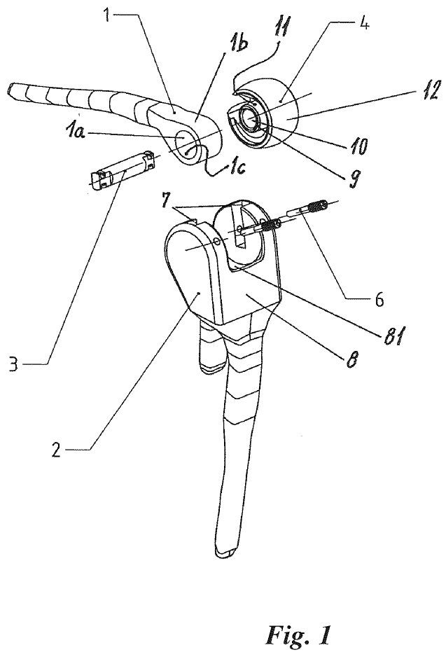

FIG. 1 is an exploded perspective view of a preferred embodiment of the elbow prosthesis implant according to the invention, and the

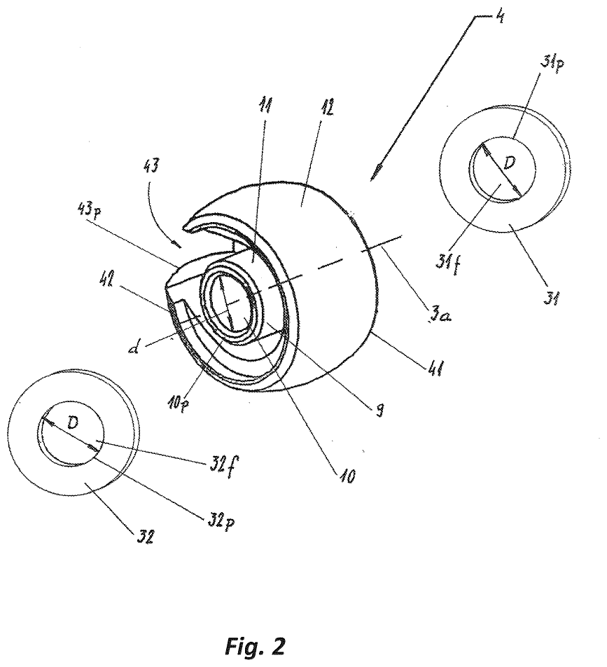

FIG. 2 shows an advantageous embodiment of the external cover shown in FIG. 1 in enlarged view.

FIG. 1 shows an exploded perspective view of a preferred embodiment of the elbow prosthesis implant according to the invention. The elbow prosthesis implant comprises a forearm stem 1 with a head 1b provided with a hole 1a and an upper arm stem 2. At the end of the upper arm stem 2, there is a socket 8 fixed to the upper arm shaft 2 and provided with grooves 7 suitable for gripping and fixing the shaft 3 that connect the stems 1, 2 to each other in a hinged manner. The shaft 3 can be properly fixed with locking pins 6 in the grooves 7.

The shaft 3 fits loosely into the hole 1a formed in the head 1b of the forearm stem 1, so that between the surface of the shaft 3 and the inner surface 1c of the hole 1a, there is an insert 9 having the form of a pipe section, in the shaft hole 10 of which the shaft 3 can rotate. The surface of the shaft hole 10 of the insert 9 is in continuous contact with the surface of the shaft 3, and its back surface 11 is in continuous contact with the surface 1c of the hole 1a. The phenomenon of wear on the back surface 11 occurs along the back surface 11 of the insert 9 and the surface 1c of the head 1b. Therefore, in the case of the solution according to the invention, the closed, outer cover 4 covering the head 1b is attached to the insert 9, the skirt surface 12 of which fits into the joint surface 81 formed in the socket 8 arranged on the forearm stem 2, which is flush with the skirt surface 12 of the cover 4. The outer cover 4 and the insert 9 are preferably made of UHMWPE or HxUHMWPE material, stems 1, 2 are made of a metal or metal alloy e.g. of Ti alloy known to a person skilled in the art of medical technology. By using the closed cover 4, it is possible to prevent back surface wear products 11 peeled off the surface 1c and the surface of the shaft hole 10 from escaping into the surrounding tissues.

FIG. 2 shows the build-up of the outer cover 4 of the advantageous embodiment shown in FIG. 1, in an enlarged view. In the presented expedient embodiment, the outer cover 4 has a skirt surface 12, which is the surface of a spherical segment of a sphere, from which two identical spherical calottes have been cut by two planes perpendicular to the axis line 3a of the shaft 3.

The insert 9 is arranged symmetrically around the axis line 3a in the hollow cover 4, so that the cover 4 is closed with closing covers 31, 32 along its both edges 41, 42. In this embodiment, the closing covers 31, 32 are coincident disc rings and are provided with a central hole 31f, 32f, the diameter D of which is identical with the inner diameter d of the shaft hole 10 of the insert 9. On the cover 4, there are 43 openings suitable for the outlet of the forearm stem 1.

In this advantageous embodiment, the prosthetic implant according to the invention is assembled in such a way that the edge 10p of the insert 9 is welded first e.g. to the edge 31p the closing cover 31, so that its hole 32f is aligned with the shaft hole 10 of the insert 9, then the closing cover 31 is attached to the edge 41 of the cover 4, preferably also by welding. After that, the hole 1a of the forearm stem 1 is pulled onto the back surface 11 of the insert 9, while the forearm stem 1 is inserted into the opening 43, then the edge 10p of the insert 9 is welded to the edge 32p of the closing cover 32 so that its hole 32f is aligned with the shaft hole 10 of the insert 9. The closing cover 32 is welded to the edge 42 of the cover 4. Finally, the forearm stem 1 is roughened around the opening 43 e.g., by laser or mechanical processing and welded to the 4 lids. The roughing can be realized e.g. by microgrooving the forearm stem 1 in the vicinity of the rim 43p of the opening 43. After that, the UHMWPE or HxUHMWPE material of the cover 4 is pressed against the roughened surface of the forearm stem 1, and pressed into the grooves by using ultrasonic welding, that is by simultaneous application of ultrasonic vibrations and pressure. The welding is performed in such a way that a continuous seam is formed along the entire length of the edges 41, 42 and along the entire circumferential edge 43p of the opening 43, resulting in the interior of the cover 4 is hermetically isolated from its external environment.

Advantageous embodiments can also be used, where the lid 4 and one of the closing lids 31, 32, as well as the insert 9, if applicable, are formed from a single piece. In this case, by drawing the hole 1a of the forearm stem 1 to the back surface 11 of the insert 9, it is necessary only to weld the other closing cover 31,32 to cover 4 and the edge of the opening 43 to the forearm stem 1.

In a further, not illustrated embodiment, the cover 4 can be made of two elements cut by a plane perpendicular to the axis line 3a or being at an acute angle to the axis line 3a and passing through the skirt surface 12, and of the insert 9, but in this case the occurrent remnants of the seam to be made on the outer surface 12 can worsen friction conditions between the skirt surface 12 and the joint surface 81.

The advantage of the elbow prosthesis implant according to the invention compared to solutions according to the state of the art is that the forearm stem 1 and the insert 9 are irreversibly fixed to each other, minimizing the micro-movements between the insert 9 and the back surface 11 leading to wear on the back surface 11, thus the production of wear products on the back is essentially eliminated, and as a result of the hermetic isolation of the back surface 11 achieved by ultrasonic welding, shivers resulting from possible wear of the back surface 11 are also prevented from leaving the prosthesis, while the design of the cover 4 is advantageously such that the ultrasonic welding does not affect the main load surfaces of the joint.

1. An elbow prosthesis implant having a forearm stem (1) formed with a head (1b) having a hole (1a), an upper arm stem (2), and a shaft (3) fixed to the upper arm stem (2) and passing through the hole (1a) of the forearm stem (1) as well as hingedly connecting the stems (1,2) to each other, and an insert (9) in the form of a pipe section is arranged between the shaft (3) and the inner surface (1c) of the hole (1a), the insert having an axial hole (10) receiving the shaft (3), comprising an outer cover (4) covering the head (1b) is attached to the insert (9), on which a first covering plate (31) with a first hole (31f) and a second covering plate (32) with a second hole (32f) both having the same diameter (D) as the diameter (d) of the axial hole (10) of the insert (9) formed as a pipe section are oppositely arranged, and a forearm stem (1) opening (43) is formed, and the flange (31p,32p) of the holes (31f,32f) is welded to the flange (10p) of the axial hole (10), the flange (43p) of the opening (43) of the forearm stem (1) is welded to the forearm stem (1) along the respective flanges (31p,32p,10p,43p) by a continuous seam.

2. (canceled)

3. The implant according to claim 1, comprising the first covering plate (31) and the second covering plate (32) are disc-shaped elements.

4. The implant according to claim 1, wherein the shaft (3) is fixed in shaft grooves (7) formed on the upper arm stem (2).

5. The implant according to claim 3, wherein the shaft (3) is fixed with a locking pin (6) in the shaft grooves (7) formed on the upper arm stem (2).

6. An implant according to claim 1, wherein the outer cover (4) and the insert (9) are made of UHMWPE or HxUHMWPE material, and the stems (1,2) are made of a medical metal alloy.

7. An implant according to claim 1, wherein a first skirt surface (12) is formed on the outer cover (4), which is in contact with a joint surface (81) of the upper arm stem (2) being congruent and parallel to the first skirt surface (12).
